(19) United States
(12) Patent Application Publication
(10) Pub. No.: US 2026/0351413 A1
(43) Pub. Date: Nov. 5, 2026

(54) METHOD AND SYSTEM FOR MULTI-MODAL DATA ANALYSIS

(71) Applicant: Qure.ai Technologies Private Limited, Mumbai (IN)

(72) Inventors: Ayushi Mahendra, Mumbai (IN); Bunty Kundnani, Mumbai (IN); Sri Anusha Matta, Mumbai (IN)

(21) Appl. No.: 19/664,856

(22) Filed: Apr. 30, 2026

(30) Foreign Application Priority Data
May 2, 2025 (IN) 202521042841

(51) Int. Cl. G16H 50/20; G16H 10/60; G16H 30/40

(52) U.S. Cl. CPC G16H 50/20; G16H 10/60; G16H 30/40

(57) ABSTRACT

The present subject matter relates to a system (100) for multi-modal data analysis. The system (100) receives a plurality of information associated with one or more users. Further, the system (100) pre-processes each of the plurality of information associated with the one or more users. Furthermore, the system (100) analyses the pre-processed plurality of information to detect one or more abnormalities using a plurality of machine learning models. Additionally, the system (100) generates one or more confidence scores by each of the plurality of machine learning models based on analyzing the pre-processed plurality of information. Moreover, the system (100) provides a multi-modal analysis report based on integrating each of the one or more confidence scores generated by each of the plurality of machine learning models. Overall, the system (100) facilitates an efficient and comprehensive approach for multi-modal data analysis.

### CROSS-REFERENCE TO RELATED APPLICATIONS AND PRIORITY

The present application claims priority from Indian Patent Application no. 202521042841 filed on May 02, 2025.

### FIELD OF INVENTION

The present invention, in general, relates to the field of data analysis and more particularly, relates to a method and a system for multi-modal data analysis.

### BACKGROUND OF THE INVENTION

This section is intended to introduce the reader to various aspects of art, which may be related to various aspects of the present disclosure that are described or claimed below. This discussion is believed to be helpful in providing the reader with background information to facilitate a better understanding of the various aspects of the present disclosure. Accordingly, it should be understood that these statements in this background section are to be read in this light, and not as admissions of prior art. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

Conventional diagnostic systems primarily focus on analyzing a single type of patient data, such as radiographic images or clinical history, to detect medical conditions. While these methods provide valuable insights, they suffer from several limitations. Traditional diagnostic tools operate in silos medical imaging, clinical records, and symptom assessments are often reviewed separately, leading to a fragmented diagnostic process. This disjointed approach increases the likelihood of missing key correlations between different data points, which are crucial for comprehensive disease detection. Moreover, early-stage symptoms such as gait abnormalities, facial distress, or respiratory sounds often go unnoticed by imaging-based diagnostic tools, resulting in delayed or inaccurate diagnoses.

Furthermore, conventional diagnostic models rely heavily on isolated data analysis, making them susceptible to diagnostic bias and inefficiencies. For instance, radiographic imaging tools primarily assess structural abnormalities but lack the capability to incorporate real-time physical and auditory symptoms into their evaluations. Similarly, AI-based image analysis tools focus solely on visual cues while disregarding clinical history, symptoms, or risk factors, leading to potential misdiagnoses. The absence of multi-modal data integration also contributes to the time-consuming nature of traditional diagnosis, as healthcare professionals must manually review and cross-reference multiple reports, increasing workload and the potential for human error.

Another limitation of existing diagnostic systems is their inability to assess respiratory and vocal symptoms through sound analysis. Conditions such as lung diseases, Parkinson’s disease, and other neurological disorders often present early indicators in the form of coughs, breathing patterns, or voice changes. However, conventional diagnostic tools lack the capability to analyze such audio cues, reducing their effectiveness in early disease detection. Additionally, many AI-based tools require sequential or manual interpretation of different data sources, further delaying the diagnostic process.

The limitations of traditional systems become even more apparent in remote diagnostics and telemedicine. Most existing diagnostic methods are not designed for remote patient assessments, making it challenging for healthcare professionals to evaluate physical symptoms or sound-based clues during virtual consultations. This limitation is particularly problematic for patients in remote or resource-limited areas who lack immediate access to specialized healthcare facilities. Additionally, single-modality diagnostic tools introduce biases by relying on limited data inputs, which may skew results or overlook subtle disease indicators.

Current AI-driven diagnostic models primarily operate on structured imaging data or unstructured textual information, but they struggle with multi-modal data interpretation. While medical imaging AI tools can detect structural abnormalities, they fail to correlate imaging findings with clinical symptoms, patient history, and real-time physical assessments. Conversely, language models are proficient at processing textual data but lack the ability to analyze structured medical images or physiological symptoms. This gap in multi-modal integration highlights the need for an advanced diagnostic system that combines various data sources including medical images, clinical records, patient videos, and audio recordings to provide a holistic and accurate diagnosis.

Additionally, traditional diagnostic tools lack the ability to personalize patient assessments by analyzing historical and real-time data simultaneously. Patients with chronic conditions such as lung diseases or Parkinson’s diseases require continuous monitoring of disease progression, but conventional systems fail to track subtle changes in symptoms over time. This results in delayed interventions and suboptimal treatment planning. Moreover, the reliance on single-modality data makes it difficult to detect early-stage diseases where minor physiological changes, rather than clear structural abnormalities, serve as primary indicators.

Given these challenges, there is a growing need for a comprehensive diagnostic solution that integrates multi-modal data sources. A system that performs independent, parallel data analysis while correlating insights across different modalities would enhance diagnostic accuracy, reduce bias, and support early disease detection. Furthermore, such a system would play a crucial role in remote diagnostics and telemedicine by facilitating patient assessments beyond traditional healthcare settings. By addressing these limitations, a multi-modal AI-driven diagnostic approach can significantly improve the efficiency, accuracy, and accessibility of modern healthcare solutions.

In light of the above stated discussion, there exists a need for an improved system and a method for multi-modal data analysis.

### SUMMARY OF THE INVENTION

Before the present system and device and its components are summarized, it is to be understood that this disclosure is not limited to the system and its arrangement as described, as there can be multiple possible embodiments which are not expressly illustrated in the present disclosure. The present disclosure overcomes one or more shortcomings of the prior art and provides additional advantages discussed throughout the present disclosure. Additional features and advantages are realized through the techniques of the present disclosure. It is also to be understood that the terminology used in the description is for the purpose of describing the versions or embodiments only and is not intended to limit the scope of the present application. This summary is not intended to identify essential features of the claimed subject matter nor is it intended for use in detecting or limiting the scope of the claimed subject matter.

According to embodiments illustrated herein, a method for multi-modal data analysis is disclosed. In one implementation of the present disclosure, the method may involve various steps performed by a processor. The method may involve a step of receiving a plurality of information associated with one or more users. Further, the method may involve a step of pre-processing each of the plurality of information associated with the one or more users. Further, the method may involve a step of analyzing the pre-processed plurality of information to detect one or more abnormalities using a plurality of machine learning models. Further, the method may involve a step of generating one or more confidence scores by each of the plurality of machine learning models based on analyzing the pre-processed plurality of information. Furthermore, the method may involve a step of providing a multi-modal analysis report based on integrating each of the one or more confidence scores generated by each of the plurality of machine learning models.

According to embodiments illustrated herein, a system for multi-modal data analysis is disclosed. In one implementation of the present disclosure, the system may involve a processor and a memory. The memory is communicatively coupled to the processor. Further, the memory is configured to store one or more executable instructions. Further, the processor may be configured to receive the plurality of information associated with the one or more users. Further, the processor may be configured to pre-process each of the plurality of information associated with the one or more users. Further, the processor may be configured to analyze the pre-processed plurality of information to detect the one or more abnormalities using the plurality of machine learning models. Further, the processor may be configured to generate the one or more confidence scores by each of the plurality of machine learning models based on analyzing the pre-processed plurality of information. Furthermore, the processor may be configured to provide the multi-modal analysis report based on integrating each of the one or more confidence scores generated by each of the plurality of machine learning models.

According to embodiments illustrated herein, there is provided a non-transitory computer-readable storage medium having stored thereon, a set of computer-executable instructions causing a computer comprising one or more processors to perform various steps. The steps may involve receiving the plurality of information associated with the one or more users. Further, the steps may involve pre-processing each of the plurality of information associated with the one or more users. Further, the steps may involve analyzing the pre-processed plurality of information to detect the one or more abnormalities using the plurality of machine learning models. Further, the steps may involve generating the one or more confidence scores by each of the plurality of machine learning models based on analyzing the pre-processed plurality of information. Furthermore, the steps may involve providing the multi-modal analysis report based on integrating each of the one or more confidence scores generated by each of the plurality of machine learning models.

The foregoing summary is illustrative only and is not intended to be in any way limiting. In addition to the illustrative aspects, embodiments, examples, and features described above, further aspects, embodiments, and features will become apparent by reference to the drawings and the following detailed description.

### BRIEF DESCRIPTION OF DRAWINGS

The detailed description is described with reference to the accompanying figures. In the figures, same numbers are used throughout the drawings to refer like features and components. Embodiments of a present disclosure will now be described, with reference to the following diagrams below wherein:

FIG. 1 illustrates a block diagram describing a system (100) for multi-modal data analysis, in accordance with at least one embodiment of present subject matter.

FIG. 2 illustrates a block diagram showing an overview of various components of an application server (101) configured for multi-modal data analysis, in accordance with at least one embodiment of present subject matter.

FIG. 3 illustrates a flowchart describing a method (300) for multi-modal data analysis, in accordance with at least one embodiment of present subject matter.

FIG. 4 illustrates a sequential flow diagram (400) illustrates a method for multi-modal data analysis, in accordance with an embodiment of present subject matter, and

FIG. 5 illustrates a block diagram (500) of an exemplary computer system (501) for implementing embodiments consistent with the present subject matter.

It should be noted that the accompanying figures are intended to present illustrations of exemplary embodiments of the present disclosure. These figures are not intended to limit the scope of the present disclosure. It should also be noted that accompanying figures are not necessarily drawn to scale.

### DETAILED DESCRIPTION OF THE INVENTION

Reference throughout the specification to “various embodiments,” “some embodiments,” “one embodiment,” or “an embodiment” means that a particular feature, structure, or characteristic described in connection with the embodiment is included in at least one embodiment. Thus, appearances of the phrases “in various embodiments,” “in another embodiments,” “in some embodiments,” “in one embodiment,” or “in an embodiment” in places throughout the specification are not necessarily all referring to the same embodiment. Furthermore, the features, structures or characteristics may be combined in any suitable manner in one or more embodiments.

The words "comprising," "having," "containing," and "including," and other forms thereof, are intended to be equivalent in meaning and be open ended in that an item or items following any one of these words is not meant to be an exhaustive listing of such item or items or meant to be limited to only the listed item or items. It must also be noted that, the singular forms "a," "an," and "the" include plural references unless the context clearly dictates otherwise. Although any methods similar or equivalent to those described herein can be used in the practice or testing of embodiments of the present disclosure, the exemplary methods are described. The disclosed embodiments are merely exemplary of the disclosure, which may be embodied in various forms.

The terminology “one or more medical images” and “medical images” has the same meaning and are used alternatively throughout the specification. Further, the terminology “one or more abnormalities” and “abnormalities” has the same meaning and are used alternatively throughout the specification. Further, the terminology “clinical information”, and “clinical data” has the same meaning and are used alternatively throughout the specification. Further, the terminology “video information”, “video recording” and “video data” has the same meaning and are used alternatively throughout the specification. Further, the terminology “audio information”, “audio data” and “audio recording” has the same meaning and are used alternatively throughout the specification. Further, the deep learning techniques are used for processing the one or more medical images. Further, the deep learning techniques corresponds to at least one of Resnet-18, Se-Resnet-18, Se-ResNeXt50, or a combination thereof. Further, large language models (LLMs) are used for analyzing the clinical information to extract contextual information. Further, the LLMs corresponds to at least one of individual generative pre-trained architecture, embodiment of other language models or a combination thereof. Further, one or more vision language models (VLMs) are used to perform either motion analysis or frame selection on the video information to detect physical symptoms. Further, the VLMs corresponds to at least one of multimodal attention mechanisms, cross-modal alignment and integration, context-aware analysis, video-based VLMs, or a combination thereof. Further, the automatic sound recognition (ASR) technique is used to extract audio features from the audio information to identify respiratory characteristics of a user. Further, the ASR technique corresponds to at least one of spectrogram-based deep learning models, time-series & frequency analysis models, or a combination thereof.

The present disclosure relates to a system for multi-modal data analysis. The system comprises a processor and a memory storing one or more executable instructions that enable the processor to receive a plurality of information associated with one or more users, pre-process each piece of the information, and analyze the pre-processed data to detect one or more abnormalities using a plurality of machine learning models. In an embodiment, the processor generates one or more confidence scores from each machine learning model based on the analysis and provides a comprehensive multi-modal analysis report by integrating these confidence scores. This approach enhances diagnostic precision and efficiency by leveraging automated data processing and advanced machine learning techniques, thus supporting early abnormality detection.

To address the limitations of conventional diagnostic solutions, the disclosed system integrates multi-modal data sources such as medical imaging, clinical history, patient videos, and respiratory sounds to enable real-time analysis across diverse modalities. Unlike traditional methods that rely on singular, sequential data analysis, the disclosed system performs independently, parallel analyses and correlates the insights to reduce bias and improve diagnostic accuracy. This multi-modal, AI-driven approach not only facilitates early disease detection and remote diagnostics but also enhances overall accessibility and reliability of modern healthcare solutions, particularly in resource-limited settings.

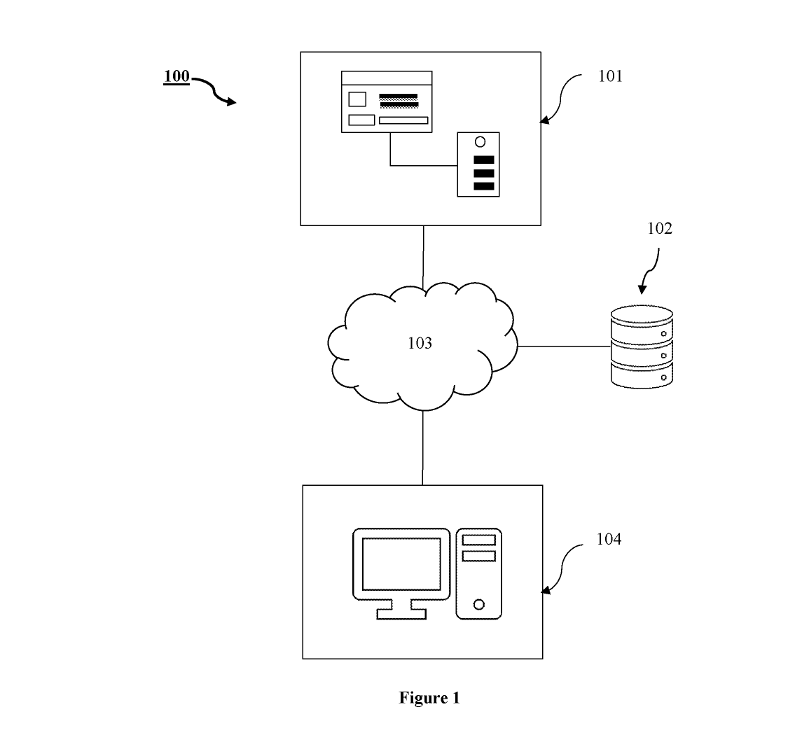

Referring to FIG. 1 is a block diagram that illustrates a system (100) for multi-modal data analysis, in accordance with at least one embodiment of the present subject matter. The system (100) typically comprises an application server (101), a database server (102), a communication network (103), and a user computing device (104). The application server (101), the database server (102), and the user computing device (104) are typically communicatively coupled with each other via the communication network (103). In an embodiment, the application server (101) may communicate with the database server (102), and the user computing device (104) using one or more protocols such as, but not limited to, Hypertext Transfer Protocol (HTTP), Transmission Control Protocol/Internet Protocol (TCP/IP), Wireless Application Protocol (WAP), RF mesh, Bluetooth Low Energy (BLE), and the like, to communicate with one another.

In an embodiment, the database server (102) may refer to a computing device that may be configured to store, manage, and retrieve data associated with the multi-modal data analysis system. The database server (102) may include a centralized repository specifically configured for storing and maintaining a structured repository of the one or more X-ray images, the clinical information, video information, audio information, one or more confidence scores, imaging score, audio score, vision score, multi-modal analysis report, and corresponding reports. Additionally, the centralized repository may also store parameters relating to the one or more deep learning techniques, training datasets, and historical diagnostic records, facilitating continuous learning and improvement of the automated multi-modal data analysis system. The database server (102) enables efficient data retrieval and integration, ensuring seamless processing of a clinical text using one or more large language models, processing the one or more medical images utilizing one or more deep learning techniques, performing either motion analysis or frame selection on the video information to detect physical symptoms utilizing one or more vision language models (VLMs ) and extracting audio features from the audio information to identify respiratory characteristics of the user using an automatic sound recognition (ASR) technique. In an alternative embodiment, the processing of the clinical text or information may be performed utilizing the one or more VLMs. By providing a scalable and secure infrastructure, the database server (102) supports real-time access to the plurality of information, allowing healthcare professionals to make informed diagnostic and treatment decisions. Moreover, the database server (102) may refer to the computing device configured to perform a variety of database operations essential for multi-modal data analysis. Further, the centralized repository may be further configured to perform database operations, such as storing, identifying, retrieving, and logging the analysis of the plurality of information from each model. In an exemplary embodiment, logging may correspond to maintaining the clinical information including at least one of user history, risk factor, symptoms, bullae characteristics, typical anatomical changes, age, smoking history, personalized risk profile, clinical guidelines, previous diagnosis notes, family history of lung disease, video data of the patient, audio recording of the patients or a combination of the same. Examples of database operations may include, but are not limited to, storing, retrieving, identifying, managing, and logging data for multi-modal data analysis. In an embodiment, the database server (102) may include hardware and software capable of being realized through various technologies, such as, but not limited to, Microsoft SQL Server, Oracle, IBM DB2, Microsoft Access, PostgreSQL, MySQL, SQLite, or distributed database technologies. The database server (102) may also be configured to utilize the application server (101) for storage and retrieval of data required for multi-modal data analysis.

A person with ordinary skills in art will understand that the scope of the disclosure is not limited to the database server (102) as a separate entity. In an embodiment, the functionalities of the database server (102) can be integrated into the application server (101) or into the user computing device (104).

In an embodiment, the application server (101) may refer to a computing device or a software framework hosting an application or a software service. In an embodiment, the application server (101) may be implemented to execute procedures such as, but not limited to, programs, routines, or scripts stored in the database server (102) for supporting the hosted application or the software service. In an embodiment, the hosted application or the software service may be configured to perform one or more predetermined operations. The application server (101) may be realized through various types of application servers such as, but are not limited to, a Java application server, a .NET framework application server, a Base4 application server, a PHP framework application server, or any other application server framework.

In an embodiment, the application server (101) application may be configured to utilize the database server (102) and the user computing device (104) in conjunction for multi-modal data analysis. In an implementation, the application server (101) corresponds to a computing system that facilitates the coordination and processing of data between the database server (102) and the user computing device (104). The application server (101) manages the flow of data by retrieving relevant clinical information, the medical images, video information and the audio information from the database server (102) and then communicates with the user computing device (104) to present the processed results to the user or the healthcare professional. The application server (101) also hosts the necessary software applications and deep learning techniques for automated analysis, classifying presence and location of the one or more abnormalities in the one or more medical images, correlating the clinical information with the one or more medical images and the video information to detect the physical symptoms of the one or more abnormalities, and providing reports. By acting as an intermediary between the database server (102) and user interfaces, the application server (101) ensures smooth data integration, efficient processing, and accurate multi-modal data analysis to support clinical decision-making. Further, by leveraging the one or more models to process the plurality of information parallelly and by integrating all the models to provide contextual insights, the application server (101), coupled with the database server (102), ensures the accurate processing of the plurality of information, such as patient history, symptoms, and risk factors.

In an embodiment, the application server (101) may be configured to receive a plurality of information associated with one or more users. In an embodiment, the one or more inputs may include at least one of the medical images, the clinical information, the video information, the audio information, or a combination thereof, associated with the one or more users.

In an embodiment, the application server (101) may be configured to pre-process each of the plurality of information associated with the one or more users.

In an embodiment, the application server (101) may be configured to analyze the pre-processed plurality of information to detect one or more abnormalities, using a plurality of machine learning models.

In an embodiment, the application server (101) may be configured to generate one or more confidence scores by each of the plurality of machine learning models based on analyzing the pre-processed plurality of information.

In an embodiment, the application server (101) may be configured to provide a multi-modal analysis report based on integrating each of the one or more confidence scores generated by each of the plurality of machine learning models.

In an embodiment, the communication network (103) may correspond to a communication medium through which the application server (101), the database server (102), and the user computing device (104) may communicate with each other. Such communication may be performed in accordance with various wired and wireless communication protocols. Examples of such wired and wireless communication protocols include, but are not limited to, Transmission Control Protocol and Internet Protocol (TCP/IP), User Datagram Protocol (UDP), Hypertext Transfer Protocol (HTTP), Wireless Application Protocol (WAP), File Transfer Protocol (FTP), ZigBee, EDGE, infrared IR), IEEE 802.11, 802.16, 2G, 3G, 4G, 5G, 6G, 7G cellular communication protocols, and/or Bluetooth (BT) communication protocols. The communication network (103) may either be a dedicated network or a shared network. Further, the communication network (103) may include a variety of network devices, including routers, bridges, servers, computing devices, storage devices, and the like. The communication network (103) may include, but is not limited to, the Internet, intranet, a cloud network, a Wireless Fidelity (Wi-Fi) network, a Wireless Local Area Network (WLAN), a Local Area Network (LAN), a cable network, the wireless network, a telephone network (e.g., Analog, Digital, POTS, PSTN, ISDN, xDSL), a telephone line (POTS), a Metropolitan Area Network (MAN), an electronic positioning network, an X.25 network, an optical network (e.g., PON), a satellite network (e.g., VSAT), a packet-switched network, a circuit-switched network, a public network, a private network, and/or other wired or wireless communications network configured to carry data.

In an embodiment, the user computing device may comprise one or more processors and one or more memory. The one or more memory may store computer-readable instructions that are executable by the one or more processors to interact with the database server (102) and the application server (101) for multi-modal data analysis. These instructions enable the computing device to receive and display the processed results from the application server (101), including medical images, clinical information, audio information, video information and combined report. The user computing device facilitates the presentation of the processed data, including the clinical information, the medical images, audio information, video information and multi-modal analysis results, to the user or the healthcare professional. The user computing device allows for seamless communication with the application server (101) to receive the multi-modal analysis reports, enabling users to review and interpret the results. Additionally, the user computing device supports the execution of various user interface applications, making it easier to access, visualize, and understand the insights provided by the system. Through this integration, the user computing device plays an important role in ensuring that healthcare professionals are able to make well-informed decisions based on real-time monitoring of the input medical images, video information, audio information along with the clinical information using one or more machine learning models.

The system (100) can be implemented using hardware, software, or a combination of both, which includes using where suitable, one or more computer programs, mobile applications, or “apps” by deploying either on-premises over the corresponding computing terminals or virtually over cloud infrastructure. The system (100) may include various micro-services or groups of independent computer programs which can act independently in collaboration with other micro-services. The system (100) may also interact with a third-party or external computer system. Internally, the system (100) may be the central processor of all requests for transactions by the various actors or users of the system. A critical attribute of the system (100) is that the system seamlessly integrates multi-modal data analysis to detect abnormalities with high accuracy. By receiving a plurality of information associated with one or more users, pre-processing the data, and analyzing it using one or more machine learning models, the system ensures precise and efficient anomaly detection. The system’s ability to generate confidence scores from each machine learning model and integrate them into a comprehensive multi-modal analysis report enhances decision-making. This automated analysis enables a holistic approach to data-driven insights, improving the overall efficiency and effectiveness of abnormality detection across various domains.

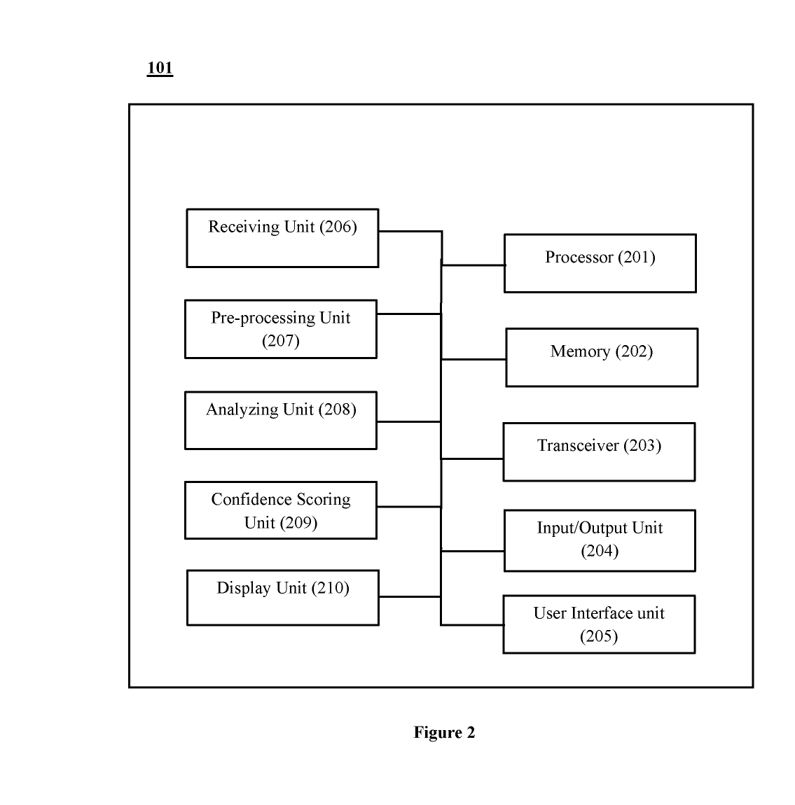

Now referring to FIG. 2, illustrate a block diagram showing an overview of various components of the application server (101) configured for the multi-modal data analysis, in accordance with at least one embodiment of the present subject matter. FIG. 2 is explained in conjunction with elements from FIG. 1. In an embodiment, the application server (101) includes a processor (201), a memory (202), a transceiver (203), an input/output unit (204), a user interface unit (205), a receiving unit (206), a pre-processing unit (207), an analyzing unit (208), a confidence scoring unit (209), and a display unit (210). The processor (201) may be communicatively coupled to the memory (202), the transceiver (203), the input/output unit (204), the user interface unit (205), the receiving unit (206), the pre-processing unit (207), the analyzing unit (208), the confidence scoring unit (209), and the display unit (210). The transceiver (203) may be communicatively coupled to the communication network (103) of the system (100).

In an embodiment, the system (100) provides a comprehensive solution for multi-modal data analysis by integrating advanced artificial intelligence (AI) and machine learning techniques with medical imaging, clinical data, video data and audio data. The system (100) processes the medical images, the clinical information, the video information and the audio information to provide an insight and findings on the one or more abnormalities. The system (100) further provides a detailed report to healthcare professionals, offering insights into the severity of the disease and personalized confidence scores. Through its automated analysis, the system (100) enables early detection, allowing for timely intervention and improved patient outcomes even if the patient is not physically with the healthcare professionals (i.e. telemedicine). The seamless integration of clinical imaging and clinical data, coupled with real-time processing capabilities, ensures that healthcare providers are able to make informed decisions based on accurate, data-driven insights, thereby optimizing the detection of the abnormalities.

The processor (201) comprises suitable logic, circuitry, interfaces, and/or code that may be configured to execute a set of instructions stored in the memory (202), and may be implemented based on several processor technologies known in the art. The processor (201) works in coordination with the memory (202), the transceiver (203), the input/output unit (204), the user interface unit (205), the receiving unit (206), the pre-processing unit (207), the analyzing unit (208), the confidence scoring unit (209), and the display unit (210) for multi-modal data analysis. Examples of the processor (201) include, but not limited to, a standard microprocessor, microcontroller, central processing unit (CPU), an X86-based processor, a Reduced Instruction Set Computing (RISC) processor, an Application- Specific Integrated Circuit (ASIC) processor, and a Complex Instruction Set Computing (CISC) processor, distributed or cloud processing unit, state machines, logic circuitries, and/or any devices that manipulate signals based on operational instructions and/or other processing logic that accommodates the requirements of the present invention.

The memory (202) comprises suitable logic, circuitry, interfaces, and/or code that may be configured to store the set of instructions, which are executed by the processor (201). Preferably, the memory (202) is configured to store one or more programs, routines, or scripts that are executed in coordination with the processor (201). Additionally, the memory (202) may include any computer-readable medium or computer program product known in the art including, for example, volatile memory, such as static random-access memory (SRAM) and dynamic random-access memory (DRAM), and/or non-volatile memory, such as read-only memory (ROM), erasable programmable ROM, a Hard Disk Drive (HDD), flash memories, Secure Digital (SD) card, Solid State Disks (SSD), optical disks, magnetic tapes, memory cards, virtual memory and distributed cloud storage. The memory (202) may be removable, non-removable, or a combination thereof. Further, the memory (202) may include routines, programs, objects, components, data structures, etc., which perform particular tasks or implement particular abstract data types. The memory (202) may include programs or coded instructions that supplement the applications and functions of the system (100). In one embodiment, the memory (202), amongst other things, serves as a repository for storing data processed, received, and generated by one or more of the programs or the coded instructions. In yet another embodiment, the memory (202) may be managed under a federated structure that enables the adaptability and responsiveness of the application server (101).

The transceiver (203) comprises suitable logic, circuitry, interfaces, and/or code that may be configured to receive, process or transmit information, data or signals, which are stored by the memory (202) and executed by the processor (201). The transceiver (203) is preferably configured to receive, process or transmit, one or more programs, routines, or scripts that are executed in coordination with the processor (201). The transceiver (203) is preferably communicatively coupled to the communication network (103) of the system (100) for communicating all the information, data, signals, programs, routines or scripts through the communication network (103).

The transceiver (203) may implement one or more known technologies to support wired or wireless communication with the communication network (103). In an embodiment, the transceiver (203) may include but is not limited to, an antenna, a radio frequency (RF) transceiver, one or more amplifiers, a tuner, one or more oscillators, a digital signal processor, a Universal Serial Bus (USB) device, a coder-decoder (CODEC) chipset, a subscriber identity module (SIM) card, and/or a local buffer. Also, the transceiver (203) may communicate via wireless communication with networks, such as the Internet, an Intranet and/or a wireless network, such as a cellular telephone network, a wireless local area network (LAN) and/or a metropolitan area network (MAN). Accordingly, the wireless communication may use any of a plurality of communication standards, protocols and technologies, such as: Global System for Mobile Communications (GSM), Enhanced Data GSM Environment (EDGE), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11b, IEEE 802.11g and/or IEEE 802.11n), voice over Internet Protocol (VoIP), Wi-MAX, a protocol for email, instant messaging, and/or Short Message Service (SMS).

The input/output (I/O) unit (204) comprises suitable logic, circuitry, interfaces, and/or code that may be configured to receive or present information. The input/output unit (204) comprises various input and output devices that are configured to communicate with the processor (201). Examples of the input devices include but are not limited to, a keyboard, a mouse, a joystick, a touch screen, a microphone, a camera, and/or a docking station. Examples of the output devices include, but are not limited to, a display screen and/or a speaker. The I/O unit (204) may include a variety of software and hardware interfaces, for example, a web interface, a graphical user interface, and the like. The I/O unit (204) may allow the system (100) to interact with the user directly or through the user computing devices (104). Further, the I/O unit (204) may enable the system (100) to communicate with other computing devices, such as web servers and external data servers (not shown). The I/O unit (204) can facilitate multiple communications within a wide variety of networks and protocol types, including wired networks, for example, LAN, cable, etc., and wireless networks, such as WLAN, cellular, or satellite. The I/O unit (204) may include one or more ports for connecting a number of devices to one another or to another server. In one embodiment, the I/O unit (204) allows the application server (101) to be logically coupled to other user computing devices (104), some of which may be built in. Illustrative components include tablets, mobile phones, wireless devices, etc.

Further, the input/output (I/O) unit (204) may be configured to manage the exchange of data between the application server (101) and the user computing device (104), ensuring that the data is smoothly communicated between the user and the multi-modal data analysis system. The I/O unit (204) handles the transmission of the clinical information, the medical images, video information, audio information and the multi-modal analysis results, allowing the user to access real-time reports and contextual insights on the abnormality detection. In an exemplary embodiment, the I/O unit (204) ensures seamless communication by managing data encoding, decoding, and error-checking, and guarantees that the data is delivered in a timely and accurate manner. By enabling reliable and efficient data transfer, the I/O unit (204) optimizes the user experience and supports the system’s overall functionality, enabling healthcare professionals to make quick and informed decisions based on the processed multi-modal data.

Further, the user interface unit (205) may facilitate interaction between the user and the system (100) by providing the multi-modal analysis report based on a selection of a set of machine learning models from the user. The user interface unit (205) enables the user or healthcare professional to select the desired machine learning models for accessing the corresponding multi-modal analysis report. In an exemplary embodiment, the user interface unit (205) may display individual AI report detailing the findings specific to imaging, text/video, and audio analysis. Additionally, the interface unit (205) may offer a combined diagnostic report that synthesizes information from all three AI systems, emphasizing common findings and cross-modal correlations. The interface may further allow the user to review detailed results, including confidence scores, detected abnormalities, and associated insights from each modality, thereby supporting a comprehensive and informed diagnostic decision-making process. By offering an accessible and streamlined experience, the user interface unit (205) ensures that the user can efficiently interact with the system, enabling quick, informed decisions in the diagnostic and treatment process.

Further, the receiving unit (206) may be configured to receive a plurality of information associated with one or more users. In an embodiment, the plurality of information may include at least one of one or more medical images, clinical information, video information, audio information, or a combination thereof. In an exemplary embodiment, the one or more medical images may comprise at least one of radiographic images, such as X-ray images, Magnetic Resonance Imaging (MRI), Computed Tomography (CT) scans, ultrasound, or a combination thereof. Similarly, the clinical information may include at least one of user medical history, radiology reports, electronic health records (EHR), clinical notes, demographic data, diagnostic impressions, or a combination thereof. Moreover, the video information may include at least one of video recordings of the physical examination of the one or more users, video recordings during a video conference, or other pre-stored videos associated with the one or more users, while the audio information may include at least one of audio recordings of the one or more users over tele or conference calls, pre-stored audio, or combination thereof.

In one embodiment, the pre-processing unit (207) is disclosed. Further, the pre-processing unit (207) may be configured for pre-processing each of the plurality of information associated with the one or more users. Further, the pre-processing unit (207) may be configured for processing the one or more medical images. In an embodiment, processing the one or more medical images corresponds to one or more of denoising, normalization, and segmentation, where segmentation techniques prepare images for feature extraction. In an embodiment, this processing of the one or more medical images is being performed utilizing one or more deep learning techniques. Furthermore, the pre-processing unit (207) may be configured for analyzing the clinical information to extract contextual data associated with the one or more users. In an embodiment, the clinical information is analyzed by utilizing one or more large language models (LLMs) or VLMs to extract relevant details such as patient history, diagnostic impressions, clinical notes, and other pertinent data.

In addition, the pre-processing unit (207) may be configured for performing either motion analysis or frame selection on the video information to detect physical symptoms. In an embodiment, the video information is analyzed for physical symptoms including changes in posture, facial expressions indicative of distress, variations in eye blinking, or hand tremors. Further, the motion analysis or frame selection may be executed utilizing one or more vision language models (VLMs) capable of processing texts, images (e.g., X-rays, MRI, CT scans), and videos. Further, the pre-processing unit (207) may be configured for extracting audio features from the audio information to identify respiratory characteristics of the one or more users. In an embodiment, the audio features comprise at least one of frequency, amplitude or a combination thereof. Further, the extraction of audio features and identification of the respiratory characteristics is performed using an automatic sound recognition (ASR) technique, thereby detecting characteristics such as abnormal coughing, irregular breathing patterns, respiratory sounds, wheezes, crackles, stridor, and hoarseness or soreness in the throat, noted in the patient’s voice. In one embodiment, the ASR technique may correspond to Spectrogram-Based Deep Learning Models, which is configured to extract respiratory sound features (e.g. wheezes, crackles, stridor) and mapping them to possible conditions. In another embodiment, the ASR technique may correspond to Time-Series and Frequency Analysis Models configured to extract temporal features (e.g. breathing cycles, pauses) and frequency features (amplitude, pitch shifts. In summary, the pre-processing unit (207) is designed to ensure that each type of data image, text, video, and audio is optimally pre-processed for subsequent analysis. This multi-modal pre-processing approach leverages advanced deep learning, large language models, vision language models, and automatic sound recognition techniques to enhance the overall accuracy and efficiency of the data analysis process.

In one embodiment, the analyzing unit (208) of the device is disclosed. Further, the analyzing unit (208) may be configured for analyzing the pre-processed plurality of information to detect one or more abnormalities using a plurality of machine learning models. In an embodiment, the plurality of machine learning models corresponds to at least one of the one or more deep learning techniques, the one or more large language models (LLMs), the one or more vision language models (VLMs), the automatic sound recognition (ASR) technique, or a combination thereof.

In another embodiment, the confidence scoring unit (209) is disclosed. Further, the confidence scoring unit (209) may be configured for generating one or more confidence scores by each of the plurality of machine learning models based on the analysis of the pre-processed plurality of information. In an exemplary embodiment, the one or more confidence scores correspond to confidence scores generated by the one or more deep learning techniques. In another exemplary embodiment, the one or more confidence scores correspond to confidence scores generated by the one or more VLMs. In another exemplary embodiment, the one or more confidence scores correspond to confidence scores generated by the ASR technique.

Further, the analyzing unit (208) may be configured for analyzing the processed one or more medical images to detect the presence and location of the one or more abnormalities. In an embodiment, the detected abnormalities are localized on the images with contours highlighting areas of concern, and the analysis is performed using the one or more deep learning techniques. Further, the one or more deep learning techniques are configured to generate an imaging score, from the one or more confidence scores generated by the deep learning models, corresponding to the presence and the location of the one or more abnormalities in the one or more medical images. In other words, the confidence scoring unit (209) in collaboration with the one or more deep learning techniques, is configured to generate a cumulative score (i.e. imaging score), based on a weighted combination of the one or more confidence scores generated by the deep learning models.

In addition, the analyzing unit (208) may be configured for correlating the clinical information with the one or more medical images and the video information to detect physical symptoms associated with the one or more abnormalities. In an embodiment, this correlation is being performed using the one or more VLMs. The physical symptoms associated with the one or more abnormalities may correspond to symptoms such as, but not limited to, user distress, unusual gait, facial expressions indicative of pain, changes in posture, or abnormal blinking. Further, the one or more VLMs are configured to generate a vision score, from the one or more confidence scores generated by the VLMs, corresponding to the physical symptoms of the one or more abnormalities. In other words, the confidence scoring unit (209) in collaboration with the one or more VLMs, is configured to generate a cumulative score (i.e. vision score), based on a weighted combination of the one or more confidence scores generated by the VLMs.

Furthermore, the analyzing unit (208) may be configured for evaluating the audio features from the audio information to detect and provide insights into the nature of the detected one or more abnormalities. In an embodiment, this evaluation is performed using the ASR technique. The detecting insight are reflecting characteristics such as, but not limited to, abnormal coughing, irregular breathing patterns, respiratory sounds, or hoarseness in the patient’s voice. Further, the ASR technique may be configured to generate an audio score, from the one or more confidence scores generated by the ASR technique, corresponding to the insight of the one or more abnormalities. In other words, the confidence scoring unit (209) in collaboration with the ASR technique, is configured to generate a cumulative score (i.e. audio score), based on a weighted combination of the one or more confidence scores generated by the ASR technique.

The analyzing unit (208) thereby bridges the gap between various data modalities by integrating insights derived from images, text, videos, and sounds. This comprehensive approach enhances diagnostic accuracy, enabling early detection of conditions such as, but not limited to, Parkinson's disease, chronic respiratory issues, or lung cancer, and supports personalized patient assessments and remote diagnostics via telemedicine. In an embodiment, the confidence scoring unit (209) generates individual scores such as imaging score for image analysis, vision score for text and video analysis, audio score from audio information. In an alternative embodiment, the confidence scoring unit (209) may be configured to generate a cumulative unified score representing a unified outcome. The cumulative unified score is generated by a weighted combination of each of the imaging score, the vision score and the audio score, generated in collaboration with the plurality of machine learning models.

Furthermore, the display unit (210) may be configured to provide the multi-modal analysis report, which is generated by integrating each of the one or more confidence scores produced by the plurality of machine learning models. In an embodiment, the multi-modal analysis report comprises a unified outcome represented by the cumulative unified score, as well as separate findings corresponding to the individual abnormalities detected by each machine learning model.

In an embodiment, the multi-modal analysis report comprises both separate findings corresponding to the one or more abnormalities detected by each machine learning model and a unified outcome generated from the cumulative score, thereby providing a comprehensive confidence level for the final diagnosis. Further, the parallel analysis is conducted by the Deep Learning technique, VLM, and ASR ensures that each data modality is independently evaluated, with each component contributing its respective confidence score. Further, the evaluated individual confidence score by each machine learning model corresponds to imaging score, vision score, audio score, or combination thereof. The combined diagnostic report, which integrates these individual scores into a cumulative score, offers clinicians a holistic view of the patient’s condition, enabling more accurate and timelier decisions.

In an exemplary embodiment, the one or more confidence scores, on a scale from 0 to 1, indicate the likelihood of the corresponding features being indicative of a potential condition or abnormality. Further, 0 represents no confidence in the abnormality being present and 1 indicates high confidence in the detection and localization of the abnormality.

In an embodiment, the cumulative score is generated based on a weighted combination of the individual confidence scores, providing an at-a-glance overall assessment of the device’s diagnostic output. The display unit (210) may present individual scores and detailed analytical reports for deep learning/AI, Vision Language Model (VLM), and Automatic Sound Recognition (ASR), allowing users to view the specific contributions of imaging, text/video, and audio analysis. By offering both individual and cumulative scores, the display unit (210) ensures transparency in the diagnostic process, enabling healthcare professionals to understand how each modality contributes to the final diagnosis and to make well-informed clinical decisions.

In an embodiment, the cumulative score is generated based on a weighted combination of the individual confidence scores, providing an at-a-glance overall assessment of the device’s output. The deep learning technique provides the imaging score for abnormality detection in images, the Vision Language Model assesses the correlation across reports, images, and videos resulting in generating the vision score, and the Automatic Sound Recognition contributes the audio score from audio-based findings. The weights for each model ensure that the contributions of each model align with their respective diagnostic significance, with the sum of the weights equal to 1 for normalization.

In an exemplary embodiment, the system (100) enables adaptive learning, refining its algorithms based on real-world usage data and clinical feedback to improve performance over time. The system (100) adapts techniques based on the modality type and input quality to optimize analysis accuracy. Regular algorithm updates ensure alignment with the latest clinical guidelines and medical knowledge, enhancing the system’s analytical precision and reliability.

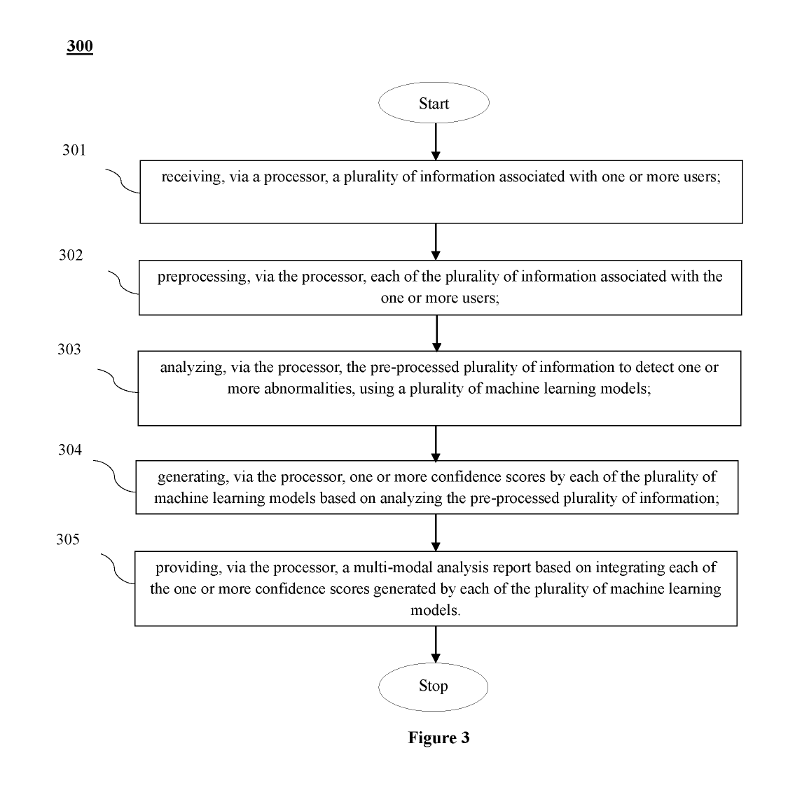

Now referring to FIG. 3, illustrates a flowchart describing a method (300) for multi-modal data analysis, in accordance with at least one embodiment of the present subject matter. The flowchart is described in conjunction with FIG. 1 and FIG. 2. The method (300) starts at step (301) and proceeds to step (305).

In operation, the method (300) may involve a variety of steps, executed by the processor (201), for multi-modal data analysis.

At step (301), the method involves receiving the plurality of information. In an embodiment, the one or more inputs may include at least one of the one or more X-ray images, the clinical information, video information, audio information associated with the one or more users.

At step (302), the method involves pre-processing each of the plurality of information associated with the one or more users.

At step (303), the method involves analyzing the pre-processed plurality of information to detect one or more abnormalities, using a plurality of machine learning models.

At step (304), the method involves generating one or more confidence scores by each of the plurality of machine learning models based on analyzing the pre-processed plurality of information.

At step (305), the method involves providing a multi-modal analysis report based on integrating each of the one or more confidence scores generated by each of the plurality of machine learning models.

### Working Example1

### Example 01: Suspected Lung Condition Diagnosis

A patient, Mr. Smith, visits a hospital with complaints of persistent cough, shortness of breath, and general fatigue. To evaluate his condition, the doctor orders a chest x-ray, records a video of Mr. Smith discussing his symptoms, and captures an audio recording of his cough for further analysis.

The receiving unit (206) receives a plurality of information associated with Mr. Smith, including medical images (chest x-ray), clinical information (medical history and doctor’s notes), video information (recording of the patient discussing symptoms), and audio information (cough sound recording). Each of these data modalities is pre-processed to optimize their quality and extract meaningful insights using the pre-processing unit (207).

### The pre-processing unit (207) performs the following pre-processing steps

Medical Image: The system processes the chest x-ray by applying denoising, normalization, and segmentation techniques utilizing deep learning models to enhance image clarity and highlight abnormalities.

Clinical Information: The system extracts contextual data from Mr. Smith’s medical history, including his smoking habits, past diagnoses, and radiology reports, using large language models (LLMs) or VLMs.

Video Information: The system applies motion analysis and frame selection using Vision Language Models (VLMs) to detect any signs of physical distress, such as laboured breathing.

Audio Feature: The system extracts audio features from the cough recording, analyzing frequency and amplitude to identify potential respiratory issues using Automatic Sound Recognition (ASR) techniques.

The analyzing unit (208) analyzes the pre-processed data using multiple machine learning models to detect abnormalities:

Medical Image Analysis: The deep learning model identifies a small lung nodule and generates an imaging confidence score based on its size, shape, and location.

Clinical Information Correlation: The VLM correlates the lung nodule with Mr. Smith’s smoking history and medical records, generating a vision confidence score that signifies a higher risk of lung cancer.

Video Analysis: The VLM detects signs of respiratory distress from the video recording, such as strained breathing and facial expressions indicative of discomfort.

Audio Analysis: The ASR model detects a deep, congested cough and hoarseness in the patient’s voice, generating an audio confidence score indicating lung congestion or airway obstruction.

The confidence scoring unit (209) generates a multi-modal analysis report by integrating findings from all modalities:

Traditional AI (Deep Learning Model): Detected a lung nodule and any other one or more abnormalities in the x-ray with a high imaging score.

VLM (Vision Language Model): Correlated the lung nodule with smoking history and detected physical distress in video analysis, assigning a vision score.

ASR (Automatic Sound Recognition): Identified abnormal cough characteristics and vocal hoarseness, assigning an audio score.

Combined Diagnostic Report: These results are synthesized into a comprehensive Combined Diagnostic Report that highlights abnormalities across all modalities, symptoms noted in the clinical records, and correlations among the various multi-modal insights. The Doctor or Mr. Smith can also access the individual model reports such as the deep learning report to view identified nodules, the VLM report to trace historical smoking records or past abnormal CT findings, and the ASR report for detailed cough analysis.

A cumulative score is generated by combining the confidence scores from the different modalities.

The display unit (210) presents a unified outcome indicating a high probability of early-stage lung cancer or severe respiratory distress. The final report includes separate findings from each model, providing the doctor with an in-depth understanding of Mr. Smith’s condition.

Based on the multi-modal analysis report, the doctor orders additional diagnostic tests, such as a CT scan and biopsy, to confirm the nature of the lung nodule and assess the severity of Mr. Smith’s condition. The integrated report, leveraging deep learning, vision-language modelling, and automatic sound recognition, enhances diagnostic accuracy, aiding clinicians in making timely and informed medical decisions.

### Working Example 2

### Example 02: Parkinson’s Disease Diagnosis

A patient, Mr. Johnson, visits a neurology clinic with complaints of tremors, difficulty walking, and slurred speech. To assess his condition, the doctor orders a CT scan, records a video of Mr. Johnson walking to analyze his gait, and captures an audio recording of his speech for further analysis.

The receiving unit (206) receives a plurality of information associated with Mr. Johnson, including medical images (CT scan), clinical information (family history and doctor’s notes), video information (recording of the patient’s gait analysis), and audio information (speech recording). Each of these data modalities is pre-processed to optimize their quality and extract meaningful insights using the pre-processing unit (207).

### The pre-processing unit (207) performs the following pre-processing steps:

Medical Image: The system processes the CT scan by applying denoising, normalization, and segmentation techniques utilizing deep learning models to enhance image clarity and detect structural abnormalities in the brain.

Clinical Information: The system extracts contextual data from Mr. Johnson’s family history, including genetic predisposition, past neurological evaluations, and clinical notes, using large language models (LLMs).

Video Information: The system applies motion analysis and frame selection using Vision Language Models (VLMs) to detect gait disturbances such as shuffling, instability, and bradykinesia.

Audio Feature: The system extracts audio features from the speech recording, analyzing frequency, amplitude, and tremor patterns to identify speech impairments associated with Parkinson’s disease using Automatic Sound Recognition (ASR) techniques.

The analyzing unit (208) analyzes the pre-processed data using multiple machine learning models to detect abnormalities:

Medical Image Analysis: The deep learning model identifies brain abnormalities indicative of Parkinson’s disease and generates an imaging confidence score based on brain region analysis.

Clinical Information Correlation: The VLM correlates the detected brain abnormalities with Mr. Johnson’s family history and clinical records, generating a vision confidence score that signifies a higher likelihood of Parkinson’s disease.

Video Analysis: The VLM detects signs of gait abnormalities from the video recording, such as reduced arm swing, slow movement, and postural instability, generating a vision confidence score.

Audio Analysis: The ASR model detects vocal tremors, speech slurring, and reduced articulation in the patient’s voice, generating an audio confidence score indicating speech impairment related to Parkinson’s disease.

The confidence scoring unit (209) generates a multi-modal analysis report by integrating findings from all modalities:

Traditional AI (Deep Learning Model): Detected structural brain abnormalities in the CT scan with a high imaging score.

VLM (Vision Language Model): Correlated brain abnormalities with family history and detected gait disturbances in video analysis, assigning a vision score.

ASR (Automatic Sound Recognition): Identified vocal tremors and speech impairment, assigning an audio score.

A cumulative score is generated by combining the confidence scores from the different modalities.

The display unit (210) presents a unified outcome indicating a high probability of Parkinson’s disease. The final report includes separate findings from each model, providing the doctor with an in-depth understanding of Mr. Johnson’s neurological condition.

In all the above examples, the system utilizes a combination of the large language model, deep learning techniques, visual language model, audio sound recognition model, clinical information, medical images, video information, audio information and the personalized confidence score for each information to detect one or more abnormalities and provide the detailed multi-modal diagnostic report to various stakeholders.

A person skilled in the art will understand that the scope of the disclosure is not limited to scenarios based on the aforementioned factors and using the aforementioned techniques and that the examples provided do not limit the scope of the disclosure.

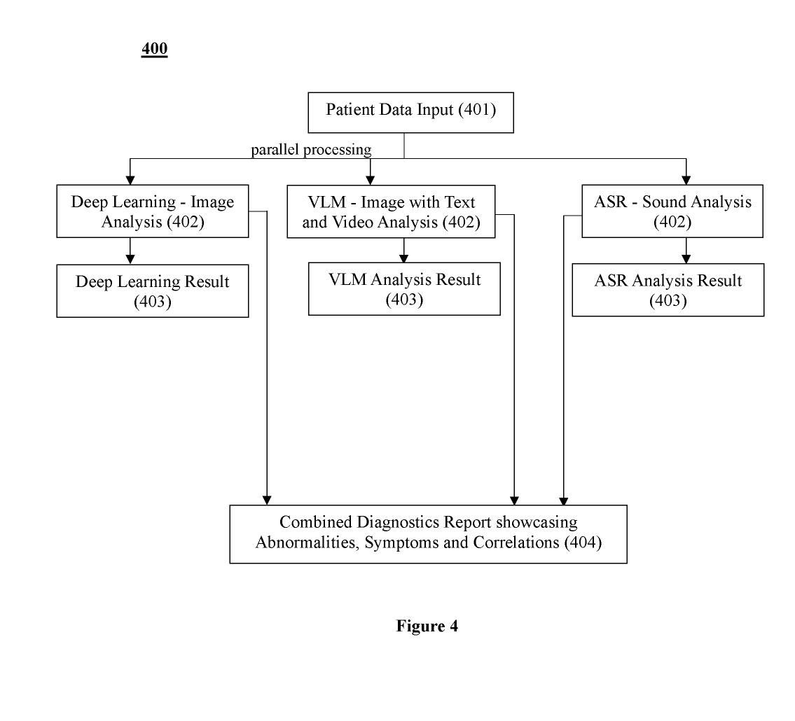

Referring to FIG. 4, a flowchart (400) illustrating a systematic method for multi-modal data analysis is presented in accordance with an embodiment of the present subject matter.

At step (401), patient data input is initiated. The process begins with the collection of various types of patient data including medical images, clinical information, video information of the patient, and audio information (e.g., cough sounds) all of which are processed in parallel.

At step (402), the collected patient data is processed by three distinct AI systems operating concurrently. Specifically, the system employs: Deep Learning AI for image analysis to detect and localize abnormalities in the medical images, a Vision Language Model (VLM) for clinical information and video information analysis, which correlates clinical reports with video information for physical symptoms such as gait, posture, and signs of discomfort and an Automatic Sound Recognition (ASR) for audio information analysis to evaluate recordings for respiratory symptoms like coughs.

At step (403), each machine learning model generates its respective analysis results. The Deep Learning AI produces an image analysis result that identifies and localizes any detected abnormalities. Concurrently, the VLM provides an analysis result that offers insights based on the correlation of clinical information, medical images, and observed physical symptoms in video information, while the ASR outputs a result detailing findings related to the analyzed sounds.

At step (404), the individual results from the three AI systems are integrated into a Combined Diagnostic Report. This comprehensive report shows abnormalities identified across all modalities, clinical symptoms noted in the reports, and the correlations between the findings from the different AI systems.

The flowchart (400) effectively illustrates the multi-modal approach of the device, demonstrating how parallel processing of patient data through distinct machine learning techniques yields both individual insights and a comprehensive diagnostic output.

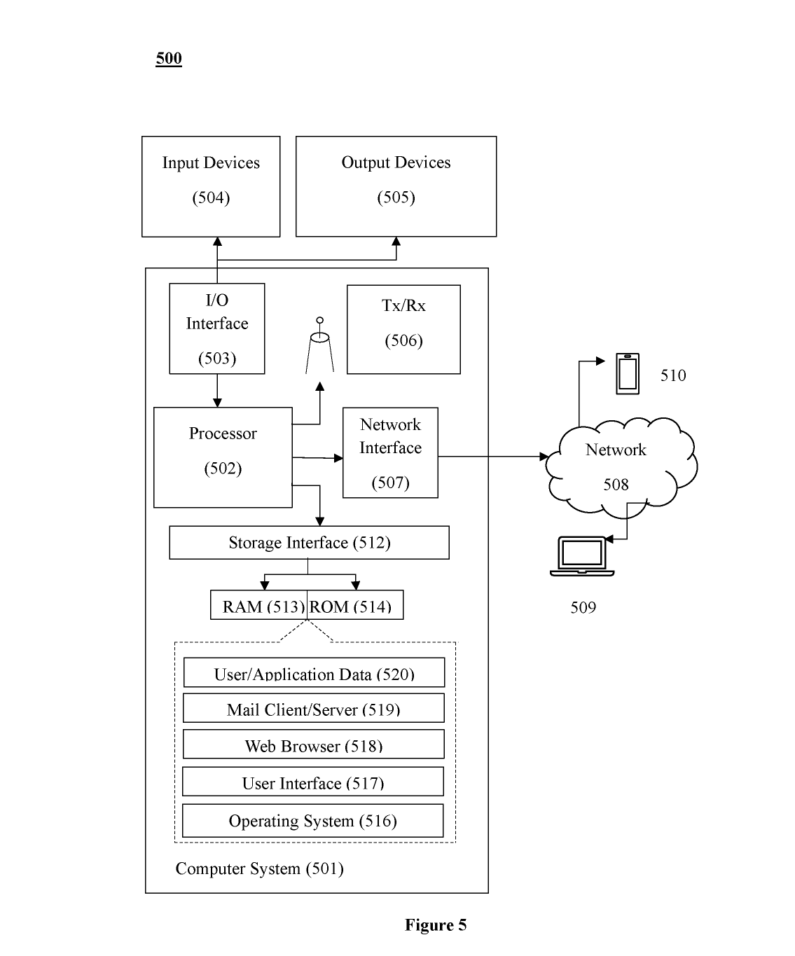

Now referring to FIG. 5 illustrates a block diagram (500) of an exemplary computer system (501) for implementing embodiments consistent with the present disclosure. Variations of computer system (501) may be used as the method for multi-modal data analysis. The computer system (501) may comprise a central processing unit (“CPU” or “processor”) (502). The processor (502) may comprise at least one data processor for executing program components for executing the user or the system generated requests. The user may include a person, a person using a device such as those included in this disclosure, or such a device itself. Additionally, the processor (502) may include specialized processing units such as integrated system (bus) controllers, memory management control units, floating point units, graphics processing units, digital signal processing units, or the like. In various implementations the processor (502) may include a microprocessor, such as AMD Athlon, Duron or Opteron, ARM’s application, embedded or secure processors, IBM PowerPC, Intel’s Core, Itanium, Xeon, Celeron or other line of processors, for example. Accordingly, the processor (502) may be implemented using mainframe, distributed processor, multi-core, parallel, grid, or other architectures. Some embodiments may utilize embedded technologies like application-specific integrated circuits (ASICs), digital signal processors (DSPs), or Field Programmable Gate Arrays (FPGAs), for example.

Processor (502) may be disposed in communication with one or more input/output (I/O) devices via I/O interface (503). Accordingly, the I/O interface (503) may employ communication protocols/methods such as, without limitation, audio, analog, digital, monoaural, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.n /b/g/n/x, Bluetooth, cellular (e.g., code-division multiple access (CDMA), high-speed packet access (HSPA+), global system for mobile communications (GSM), long-term evolution (LTE), WiMAX, or the like, for example.

Using the I/O interface (503), the computer system (501) may communicate with one or more I/O devices. For example, the input device (504) may be an antenna, keyboard, mouse, joystick, (infrared) remote control, camera, card reader, fax machine, dongle, biometric reader, microphone, touch screen, touchpad, trackball, sensor (e.g., accelerometer, light sensor, GPS, gyroscope, proximity sensor, or the like), stylus, scanner, storage device, transceiver, video device/source, or visors, for example. Likewise, an output device (505) may be a user’s smartphone, tablet, cell phone, laptop, printer, fax machine, video display (e.g., cathode ray tube (CRT), liquid crystal display (LCD), light- emitting diode (LED), plasma, or the like), or audio speaker, for example. In some embodiments, a transceiver (506) may be disposed in connection with the processor (502). The transceiver (506) may facilitate various types of wireless transmission or reception. For example, the transceiver (506) may include an antenna operatively connected to a transceiver chip (example devices include the Texas Instruments® WiLink WL1283, Broadcom® BCM4750IUB8, Infineon Technologies® X-Gold 618-PMB9800, or the like), providing IEEE 802.11a/b/g/n, Bluetooth, FM, global positioning system (GPS), and/or 2G/3G/5G/6G HSDPA/HSUPA communications, for example.

In some embodiments, the processor (502) may be disposed in communication with a communication network (508) via a network interface (507). The network interface (507) is adapted to communicate with the communication network (508). The network interface, coupled to the processor may be configured to facilitate communication between the system and one or more external devices or networks. The network interface (507) may employ connection protocols including, without limitation, direct connect, Ethernet (e.g., twisted pair 10/100/1000 Base T), transmission control protocol/internet protocol (TCP/IP), token ring, or IEEE 802.11a/b/g/n/x, for example. The communication network (508) may include, without limitation, a direct interconnection, local area network (LAN), wide area network (WAN), wireless network (e.g., using Wireless Application Protocol), or the Internet, for example. Using the network interface (507) and the communication network (508), the computer system (501) may communicate with devices such as shown as a laptop (509) or a mobile/cellular phone (510). Other exemplary devices may include, without limitation, personal computer(s), server(s), fax machines, printers, scanners, various mobile devices such as cellular telephones, smartphones (e.g., Apple iPhone, Blackberry, Android-based phones, etc.), tablet computers, eBook readers (Amazon Kindle, Nook, etc.), laptop computers, notebooks, gaming consoles (Microsoft Xbox, Nintendo DS, Sony PlayStation, etc.), or the like. In some embodiments, the computer system (501) may itself embody one or more of these devices.

In some embodiments, the processor (502) may be disposed in communication with one or more memory devices (e.g., RAM 413, ROM 414, etc.) via a storage interface (512). The storage interface (512) may connect to memory devices including, without limitation, memory drives, removable disc drives, etc., employing connection protocols such as serial advanced technology attachment (SATA), integrated drive electronics (IDE), IEEE-1394, universal serial bus (USB), fiber channel, small computer systems interface (SCSI), etc. The memory drives may further include a drum, magnetic disc drive, magneto-optical drive, optical drive, redundant array of independent discs (RAID), solid-state memory devices, or solid-state drives, for example.

The memory devices may store a collection of program or database components, including, without limitation, an operating system (516), user interface application (517), web browser (518), mail client/server (519), user/application data (520) (e.g., any data variables or data records discussed in this disclosure) for example. The operating system (516) may facilitate resource management and operation of the computer system (501). Examples of operating systems include, without limitation, Apple Macintosh OS X, UNIX, Unix-like system distributions (e.g., Berkeley Software Distribution (BSD), FreeBSD, NetBSD, OpenBSD, etc.), Linux distributions (e.g., Red Hat, Ubuntu, Kubuntu, etc.), IBM OS/2, Microsoft Windows (XP, Vista/7/8, etc.), Apple iOS, Google Android, Blackberry OS, or the like.

The user interface (517) is for facilitating the display, execution, interaction, manipulation, or operation of program components through textual or graphical facilities. For example, user interfaces may provide computer interaction interface elements on a display system operatively connected to the computer system (501), such as cursors, icons, check boxes, menus, scrollers, windows, or widgets, for example. Graphical user interfaces (GUIs) may be employed, including, without limitation, Apple Macintosh operating systems’ Aqua, IBM OS/2, Microsoft Windows (e.g., Aero, Metro, etc.), Unix X-Windows, or web interface libraries (e.g., ActiveX, Java, JavaScript, AJAX, HTML, Adobe Flash, etc.), for example.

In some embodiments, the computer system (501) may implement a web browser (518) stored program component. The web browser (518) may be a hypertext viewing application, such as Microsoft Internet Explorer, Google Chrome, Mozilla Firefox, Apple Safari, or Microsoft Edge, for example. Secure web browsing may be provided using HTTPS (secure hypertext transport protocol), secure sockets layer (SSL), Transport Layer Security (TLS), or the like. Web browsers may utilize facilities such as AJAX, DHTML, Adobe Flash, JavaScript, Java, or application programming interfaces (APIs), for example. In some embodiments the computer system (501) may implement a mail client/server (519) stored program component. The mail server (519) may be an Internet mail server such as Microsoft Exchange, or the like. The mail server may utilize facilities such as ASP, ActiveX, ANSI C++/C#, Microsoft .NET, CGI scripts, Java, JavaScript, PERL, PHP, Python, or WebObjects, for example. The mail server (519) may utilize communication protocols such as internet message access protocol (IMAP), messaging application programming interface (MAPI), Microsoft Exchange, post office protocol (POP), simple mail transfer protocol (SMTP), or the like. In some embodiments, the computer system (501) may implement a mail client (520) stored program component. The mail client (520) may be a mail viewing application, such as Apple Mail, Microsoft Entourage, Microsoft Outlook, or Mozilla Thunderbird.

In some embodiments, the computer system (501) may store user/application data (521), such as the data, variables, records, or the like as described in this disclosure. Such databases may be implemented as fault-tolerant, relational, scalable, secure databases such as Oracle or Sybase, for example. Alternatively, such databases may be implemented using standardized data structures, such as an array, hash, linked list, struct, structured text file (e.g., XML), table, or as object-oriented databases (e.g., using ObjectStore, Poet, Zope, etc.). Such databases may be consolidated or distributed, sometimes among the various computer systems discussed above in this disclosure. It is to be understood that the structure and operation of the any computer or database component may be combined, consolidated, or distributed in any working combination.

Furthermore, one or more computer-readable storage media may be utilized in implementing embodiments consistent with the present invention. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer- readable medium” should be understood to include tangible items and exclude carrier waves and transient signals, i.e., non-transitory. Examples include Random Access Memory (RAM), Read- Only Memory (ROM), volatile memory, non-volatile memory, hard drives, Compact Disc (CD) ROMs, Digital Video Disc (DVDs), flash drives, disks, and any other known physical storage media.

In light of the above-mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

Various embodiments of the disclosure provide a non-transitory computer readable medium and/or storage medium, and/or a non-transitory machine-readable medium and/or storage medium having stored thereon, a machine code and/or a computer program having at least one code section executable by a machine and/or a computer for multi-modal data analysis. The at least one code section in the application server (101) causes the machine and/or computer including one or more processors to perform the steps, which includes receiving (301), a plurality of information associated with one or more users and pre-processing (302), each of the plurality of information associated with the one or more users. Further, the step may include analyzing (303), the pre-processed plurality of information to detect one or more abnormalities, using a plurality of machine learning models. Furthermore, the step may include generating (304), one or more confidence scores by each of the plurality of machine learning models based on analyzing the pre-processed plurality of information. Additionally, the step may include providing (305), a multi-modal analysis report based on integrating each of the one or more confidence scores generated by each of the plurality of machine learning models.

Various embodiments of the disclosure encompass numerous advantages including the system and the method for multi-modal data analysis. The disclosed method and system have several technical advantages, but are not limited to the following: Enhanced Multi-Modal Data Integration: The system processes diverse data sources, including medical images, clinical history, real-time physiological signals, and audio recordings, ensuring comprehensive diagnostic assessments. Real-Time Processing for Remote Diagnosis: The system facilitates remote patient monitoring by analyzing multi-modal data in real time, enabling timely interventions and improving accessibility to healthcare in remote or resource-limited areas. Improved Diagnostic Accuracy: By integrating machine learning models to process and correlate multiple data modalities, the system enhances diagnostic accuracy, reducing the risk of misdiagnosis and missed early-stage disease indicators. Adaptable pre-processing Techniques: The pre-processing module dynamically adjusts based on input type and quality, ensuring optimal data conditioning for improved downstream analysis and interpretation. Automated Multi-Modal Report Generation: The system generates a consolidated, user-friendly diagnostic report that provides actionable insights by integrating confidence scores from multiple machine learning models. Reduced Diagnostic Bias: The correlation of structured and unstructured data, including imaging, clinical notes, and patient-reported symptoms, reduces reliance on isolated data points, thereby minimizing diagnostic bias. Enhanced Computational Efficiency: Unlike conventional methods that process different modalities sequentially, the proposed system executes concurrent multi-modal analysis, significantly reducing processing time and improving workflow efficiency.

Support for Complex Clinical Scenarios: The system extends diagnostic capabilities beyond conventional imaging-based assessments by incorporating additional health indicators, making it useful for detecting neurological, respiratory, and systemic diseases.

In summary, the technical advantages of this invention address the challenges associated with conventional data analysis in diagnostic systems, such as the limitations of single-modality data processing and lack of real-time multi-modal analysis. Traditional diagnostic tools often operate in silos, requiring healthcare professionals to manually integrate findings from disparate sources, which increases workload and the likelihood of errors. In contrast, the disclosed system concurrently processes and correlates multi-modal inputs, providing a more holistic and precise diagnosis. Additionally, conventional AI-based diagnostic models focus on either structured imaging data or unstructured text information, failing to integrate real-time physical and auditory assessments. This gap is effectively bridged by the proposed system, which enhances accuracy of the data analysis through multi-modal data fusion. The disclosed system's ability to analyze respiratory sounds, patient videos, and historical clinical data makes it particularly effective in detecting early-stage diseases that may not be evident in radiographic images alone. Furthermore, its remote diagnostic capability enables real-time assessments in telemedicine settings, improving healthcare accessibility and ensuring early intervention for patients in underserved regions. By leveraging advanced machine learning techniques and computational efficiency, the system optimizes clinical decision-making, reduces diagnostic delays, and supports more effective patient management.

The claimed invention of the system and the method for multi-modal data analysis involves tangible components, processes, and functionalities that interact to achieve specific technical outcomes. The system integrates various elements such as processors, memory, databases, matchmaking, offer calculation and informed displaying techniques to effectively perform the detection of one or more abnormalities.

Furthermore, the invention involves a non-trivial combination of technologies and methodologies that provide a technical solution for a technical problem. While individual components like processors, databases, encryption, authorization and authentication are well-known in the field of computer science, their integration into a comprehensive system for multi-modal data analysis brings about an improvement and technical advancement in the field of clinical data analysis and other related environments.

In light of the above-mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

The present disclosure may be realized in hardware, or a combination of hardware and software. The present disclosure may be realized in a centralized fashion, in at least one computer system, or in a distributed fashion, where different elements may be spread across several interconnected computer systems. A computer system or other apparatus adapted for carrying out the methods described herein may be suited. A combination of hardware and software may be a general-purpose computer system with a computer program that, when loaded and executed, may control the computer system such that the computer system carries out the methods described herein. The present disclosure may be realized in hardware that includes a portion of an integrated circuit that also performs other functions.

A person with ordinary skills in the art will appreciate that the systems, modules, and sub-modules have been illustrated and explained to serve as examples and should not be considered limiting in any manner. It will be further appreciated that the variants of the above disclosed system elements, modules, and other features and functions, or alternatives thereof, may be combined to create other different systems or applications.

Those skilled in the art will appreciate that any of the aforementioned steps and/or system modules may be suitably replaced, reordered, or removed, and additional steps and/or system modules may be inserted, depending on the needs of a particular application. In addition, the systems of the aforementioned embodiments may be implemented using a wide variety of suitable processes and system modules, and are not limited to any particular computer hardware, software, middleware, firmware, microcode, and the like. The claims can encompass embodiments for hardware and software, or a combination thereof.

While the present disclosure has been described with reference to certain embodiments, it will be understood by those skilled in the art that various changes may be made, and equivalents may be substituted without departing from the scope of the present disclosure. In addition,

many modifications may be made to adapt a particular situation or material to the teachings of the present disclosure without departing from its scope. Therefore, it is intended that the present disclosure is not limited to the particular embodiment disclosed, but that the present disclosure will include all embodiments falling within the scope of the appended claims.

We claim:

1. A method (300) for multi-modal data analysis, the method (300) comprises:
receiving (301), via a processor (201), a plurality of information associated with one or more users;
pre-processing (302), via the processor (201), each of the plurality of information associated with the one or more users;
analyzing (303), via the processor (201), the pre-processed plurality of information to detect one or more abnormalities, using a plurality of machine learning models;
generating (304), via the processor (201), one or more confidence scores by each of the plurality of machine learning models based on analyzing the pre-processed plurality of information; and
providing (305), via the processor (201), a multi-modal analysis report based on integrating each of the one or more confidence scores generated by each of the plurality of machine learning models.

2. The method (300) as claimed in claim 1, wherein the plurality of information comprises one or more medical images, clinical information, video information, audio information, and a combination thereof;
wherein the one or more medical images comprises at least one of X-ray image, Magnetic resonance imaging (MRI), Computed Tomography (CT) scan, ultrasound or a combination thereof;
wherein the clinical information comprises at least one of user medical history, radiology report, electronic health records (EHR), clinical notes, demographic data, diagnostic impression or a combination thereof;
wherein the video information comprises at least one of video recording of physical examination of the one or more users, video recording during video conference, other pre-stored video associated with the one or more users, or a combination thereof;
wherein the audio information comprises at least one of an audio recording of the one or more users over tele call or conference call, other pre-stored audio associated with the one or more users, or a combination thereof.

3. The method (300) as claimed in claim 2, wherein pre-processing (302) each of the plurality of information comprises:
processing. the one or more medical images, wherein processing the one or more medical images corresponds to one of denoising, normalization, segmentation or a combination thereof, wherein processing of the one or more medical images being performed utilizing one or more deep learning techniques;
analyzing. the clinical information to extract contextual information associated with the one or more users, wherein the clinical information is analyzed by utilizing one or more large language models (LLMs);
performing. either motion analysis or frame selection on the video information to detect physical symptoms, wherein the motion analysis or the frame selection on the video information is performed utilizing one or more vision language models (VLMs);
extracting audio features from the audio information to identify respiratory characteristics of the one or more users, wherein the audio features comprise at least one of frequency, amplitude or a combination thereof, wherein the extracting of the audio features and the identifying of the respiratory characteristics is being performed using an automatic recognition (ASR) technique.

4. The method (300) as claimed in claim 3, wherein the plurality of machine learning models corresponds to at least one of the one or more deep learning techniques, the one or more LLMs, the one or more VLMs, the ASR technique or a combination thereof.

5. The method (300) as claimed in claim 3, wherein analyzing (303) the pre-processed plurality of information comprises:
analyzing the processed one or more medical images to detect presence and location of the one or more abnormalities in the one or more medical images, wherein the analyzing of the processed one or more medical images is being performed using the one or more deep learning techniques wherein the one or more deep learning techniques generates an imaging score, from the one or more confidence scores, corresponding to the presence and the location of the one or more abnormalities in the one or more medical images;
correlating. the clinical information with the one or more medical images and the video information to detect the physical symptoms of the one or more abnormalities, wherein the correlating is being performed using the one or more VLMs wherein the physical symptoms of the one or more abnormalities corresponds to at least one of user distress, unusual gait, facial expression indicating pain, or a combination thereof, wherein the one or more VLMs generates a vision score, from the one or more confidence scores, corresponding to the physical symptoms of the one or more abnormalities;
evaluating. the audio features from the audio information to detect an insight of the one or more abnormalities, wherein the evaluating is being performed using the ASR technique, wherein the ASR techniques generate an audio score, from the one or more confidence scores, corresponding to the insight of the one or more abnormalities.

6. The method (300) as claimed in claim 1, wherein the one or more abnormalities comprises at least one of lesions, fractures, tumors or a combination thereof.

7. The method (300) as claimed in claim 5, wherein multi-modal analysis report comprises a unified outcome generated by the plurality of machine learning models, wherein a cumulative score is generated from the one or more confidence scores for generating the unified outcome, wherein the cumulative score is generated based on a weighted combination of the each of the one or more confidence scores.

8. The method (300) as claimed in claim 1, wherein multi-modal analysis report comprises separate findings corresponding to the one or more abnormalities detected by each of the plurality of machine learning models.

9. The method (300) as claimed in claim 1, comprising receiving a selection of a set of machine learning models, from a user, for accessing the multi-modal analysis report, wherein the method (300) comprises providing the multi-modal analysis report to the user based on the set of machine learning models selected by the user.

10. A system (100) for multi-modal data analysis, the system (100) comprises:
a processor (201),
a memory (202) communicatively coupled with the processor (201), wherein the memory (202) is configured to store one or more executable instructions, which cause the processor (201) to:
receive (301) a plurality of information associated with one or more users;
pre-process (302) each of the plurality of information associated with the one or more users;
analyze (303) the pre-processed plurality of information to detect one or more abnormalities, using a plurality of machine learning models;
generate (304) one or more confidence scores by each of the plurality of machine learning models based on analyzing the pre-processed plurality of information; and
provide (305) a multi-modal analysis report based on integrating each of the one or more confidence scores generated by each of the plurality of machine learning models.

11. A non-transitory computer-readable storage medium having stored thereon, a set of computer-executable instructions causing a computer comprising one or more processors to perform steps comprising:
receiving (301), a plurality of information associated with one or more users;
pre-processing (302), each of the plurality of information associated with the one or more users;
analyzing (303), the pre-processed plurality of information to detect one or more abnormalities, using a plurality of machine learning models;
generating (304), one or more confidence scores by each of the plurality of machine learning models based on analyzing the pre-processed plurality of information; and
providing (305), a multi-modal analysis report based on integrating each of the one or more confidence scores generated by each of the plurality of machine learning models.
